(19) United States
(12) Patent Application Publication
(10) Pub. No.: US 2026/0284026 A1
(43) Pub. Date: Sep. 24, 2026

(54) WATER-SOLUBLE AND DISPERSIBLE PIPERINE METAL-ALCOHOL COMPLEXES AND METHOD OF PREPARATION THEREOF

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/551,049

(22) Filed: Feb. 26, 2026

Related U.S. Application Data
(60) Provisional application No. 63/765,383, filed on Feb. 28, 2025.

(51) Int. Cl. A61K 31/4525; C07F 1/02; C07F 1/04

(52) U.S. Cl. CPC A61K 31/4525; C07F 1/02; C07F 1/04

(57) ABSTRACT

Compositions of metal alkoxide-piperinoid complexes that provide for water-soluble and/or dispersible piperine are disclosed. Methods of producing metal alkoxide-piperinoid complexes are disclosed. Methods of treatment using the metal alkoxide-piperinoid complexes are disclosed.

### RELATED APPLICATIONS

This application claims the benefit of and priority to U.S. Provisional Patent Application No. 63/765,383, filed Feb. 28, 2025, entitled “WATER-SOLUBLE AND DISPERSIBLE PIPERINE METAL-ALCOHOL COMPLEXES AND METHOD OF PREPARATION THEREOF”, the entire disclosure of which is incorporated herein by reference.

### TECHNICAL FIELD

The disclosure relates to pharmaceutical and nutraceutical formulations, specifically to the development of a water-soluble and/or dispersible form of piperine and its isomers, a compound with known health benefits and the ability to improve the oral absorption of various drugs and nutrients such as amino-acids, resveratrol, curcumin, Vitamin A and many others, especially those subjected to glucuronidation metabolism. The invention provides improved bioavailability, solubility, and versatility of piperine for use in various applications, including topical, oral, and injectable formulations.

### BACKGROUND

Piperine is a naturally occurring alkaloid compound of pepper (i.e., piper nigrum and related species, not to be confused with peppers such as chili peppers where capsaicin is the prime pungent compound) (Table 1). It is known for its spicy/pungent flavor and is used as a bioavailability enhancer of various drugs and supplements.

TABLE 1
Chemical structures of piperine and its isomers⁵

| Name of | |
|---|---|
| isomers | Chemical structure |
| Piperine (trans-trans isomer) | 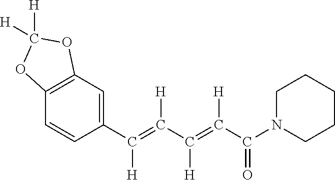 [SMILES: O=C(/C=C/C=C/c1ccc2c(c1)OCO2)N1CCCCC1] |
| Isopiperine (cis-trans isomer) | 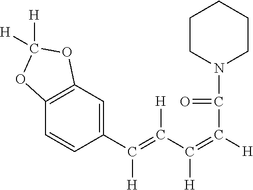 [SMILES: O=C(/C=C\C=C\c1ccc2c(c1)OCO2)N1CCCCC1] |
| chavicine (cis-cis isomer) | 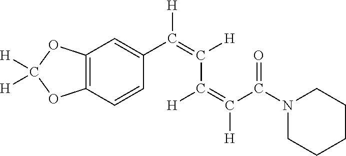 [SMILES: O=C(/C=C\C=C/c1ccc2c(c1)OCO2)N1CCCCC1] |
| Isochavicine (trans-cis isomer) | 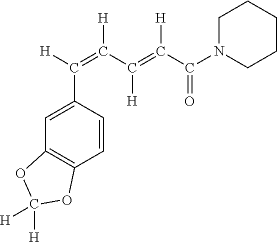 [SMILES: O=C(/C=C/C=C\c1ccc2c(c1)OCO2)N1CCCCC1] |

Various mechanisms have been proposed for the bioavailability-enhancing effects (increasing absorption of other compounds after oral administration) of piperine:
Inhibition of Glucuronidation: Piperine can inhibit the process of glucuronidation, a pathway by which the liver makes drugs and substances more water-soluble and dispersible for excretion. By slowing this process, piperine helps certain drugs stay in the body longer which leads to increased effectiveness of them.
Inhibition of Drug-Metabolizing Enzymes: Piperine inhibits cytochrome P450 enzymes, especially CYP3A4 and CYP2D6. These enzymes typically metabolize drugs in the liver and intestines. By inhibiting these enzymes, piperine slows the breakdown of drugs, allowing them to remain in the bloodstream longer and at higher concentrations.
Inhibition of P-Glycoprotein: Piperine inhibits P-glycoprotein, a membrane protein that actively pumps drugs and other compounds out of cells. P-glycoprotein is part of the body's defense system, reducing the absorption of foreign substances. By inhibiting this protein, piperine increases the amount of a drug or biologically active natural compound that can be absorbed into the bloodstream.
Increased Gastrointestinal Blood Flow: Piperine has been shown to increase blood flow in the gastrointestinal tract, enhancing the absorption of nutrients and drugs.
Enhanced Membrane Fluidity: Piperine enhances the permeability of the intestinal mucosa, making it easier for drugs and nutrients to pass through the gut lining and into the bloodstream.

However, piperine is a hydrophobic (not water-soluble and/or dispersible) compound, which limits its bioavailability and efficacy when used in per os (“by mouth” or orally administered) compositions and aqueous environments such as beverages, aqueous-based topical treatments, and injectable formulations. Piperine's low water solubility and dispersibility means it does not dissolve easily in the aqueous environment of the gastrointestinal (GI) tract. For optimal absorption to occur, a compound must be dissolved in the GI fluids. Since piperine remains largely undissolved, less of it is available for uptake into the bloodstream unless ingested with high amounts of fats. Due to its limited solubility, piperine struggles to permeate the intestinal lining effectively. Compounds need a certain degree of solubility to be absorbed passively across cell membranes, and piperine's low solubility restricts this pathway, reducing its systemic bioavailability.

Low solubility can also lead to greater exposure to enzymatic degradation in the GI tract and liver before piperine reaches systemic circulation. Compounds that are not readily absorbed tend to undergo more extensive first-pass metabolism (conversion of said compounds to their metabolites which are often inactive, before reaching the bloodstream), resulting in a smaller portion reaching circulation in an active form. Because piperine does not dissolve well, in aqueous solutions such as in water of gastrointestinal fluids its absorption rate can be erratic, leading to inconsistent levels of the compound in the bloodstream. This variability reduces its overall bioactivity, as it is harder to maintain effective concentrations. Thus, it has been reported that in humans, piperine's bioavailability is notably low, with studies indicating that only about 1-2% of an orally administered dose is absorbed into the bloodstream. For instance, a study noted that after consuming 50 mg of piperine, approximately only 0.71-0.83 mg was detectable in plasma, highlighting its limited absorption (Dludla, P. V.; Cirilli, I.; Marcheggiani, F.; Silvestri, S.; Orlando, P.; Muvhulawa, N.; Moetlediwa, M. T.; Nkambule, B. B.; Mazibuko-Mbeje, S. E.; Hlengwa, N.; et al. Bioactive Properties, Bioavailability Profiles, and Clinical Evidence of the Potential Benefits of Black Pepper (Piper nigrum) and Red Pepper (Capsicum annum) against Diverse Metabolic Complications. Molecules 2023, 28, 6569).

To address this limitation, formulations such as nano-encapsulation, micelles, or co-administration with solubility enhancers are sometimes used to improve piperine's solubility, and consequently, its bioactivity. However, none of those solutions have met commercial success or wide acceptance due to cost, difficulty in formulation, minimal results and lack of verification of in vivo activity in humans.

### SUMMARY

Disclosed is a water-soluble and/or dispersible piperine that is complexed with an alkoxide, such as lithium methoxide, lithium ethoxide, lithium propoxide, lithium butoxide, sodium methoxide, sodium ethoxide, sodium propoxide, sodium butoxide, potassium methoxide, potassium ethoxide, potassium propoxide, potassium butoxide and the like. These readily water-soluble and dispersible piperine complexes can be used in a variety of pharmaceutical, nutraceutical, dietary supplement and cosmetic applications.

Also disclosed are methods for making piperine water-soluble and/or dispersible through complexing the compound with an alkoxide, such as lithium methoxide, lithium ethoxide, lithium propoxide, lithium isopropoxide, lithium isobutoxide, lithium butoxide, sodium methoxide, sodium ethoxide, sodium propoxide, sodium isopropoxide, sodium butoxide, sodium isobutoxide, potassium methoxide, potassium ethoxide, potassium propoxide, potassium isopropoxide, potassium butoxide potassium isobutoxide and the like.

In one aspect, a method of producing the water-soluble and dispersible piperine is disclosed, which involves the solubilization of piperine in a polar, most preferably anhydrous, alcohol containing in solution a metal cation (essentially an alkoxide of said cation in solution and in the case of ethanol as a solvent, an ethoxide)-preferably produced by reaction of elemental metal with an anhydrous alcohol- and then drying out the solvent. However, premade/commercially available alkoxides can be dissolved in said alcohol, piperine dissolved in said solution, and the solution dried to form the alkoxide-piperine complex. In some embodiments, the step of dissolving piperine and/or a metabolite or derivative thereof in the alkali metal alkoxide/alcohol solution includes dissolving a piperine metabolite or derivative selected from piperic acid, a salt of piperic acid, an ester of piperic acid, a piperine salt, or a piperine ester. In certain embodiments, where piperic acid is employed, contact with an alkali metal alkoxide in an anhydrous alcohol can form an alkali metal piperate (salt) in situ, which is then carried through the same drying step under anhydrous conditions to provide a solid product that is water-dispersible and/or water-soluble.

In another aspect, hydroxides or hydrides of said metals can be dissolved in alcohols and the water be removed from the reaction by use of an insoluble dehydrating agent such as CaO or molecular sieves. The solution is then stirred/mixed till the water has been removed and the solution dried. In example embodiments, it is important for the process to take place in an anhydrous environment-even atmospheric moisture can convert an alkoxide to its alcohol and hydroxide form, whose drying out will not produce sufficiently soluble piperine alkoxide complexes. In fact, water would result in the alcohol being evaporated and left with a mixture of piperine and a metal hydroxide-which not only is not soluble and dispersible in water but carries the risk of alkalic hydrolysis of piperine. For the same reason, for optimal functionality, the products produced thus should be kept in as moisture free of an environment as can be, such as vacuum sealed, microencapsulated, in the presence of inert gases, microencapsulated by a material that water does not penetrate such as a wax, and the like.

In yet another aspect, a composition is disclosed comprising a complex of piperine and/or a metabolite or derivative thereof and an alkali metal alkoxide of an alkali metal. In certain embodiments, the alkali metal alkoxide is one of sodium ethoxide, potassium ethoxide, or lithium ethoxide. In certain embodiments, the piperine and/or a metabolite or derivative thereof is piperine. In certain embodiments, the weight ratio of alkali metal alkoxide to piperine and/or a metabolite or derivative thereof is between 1:1 to 1:1000, preferably between 0.001:1 to 1:1000, more preferably between 0.01:1 to 1:100 and even more preferably between 0.1:1 to 1:10. In certain embodiments, the weight ratio of alkali metal alkoxide to piperine and/or a metabolite or derivative thereof is between 1:1 to 1:10.

In still another aspect, the method of producing a composition is disclosed comprising: generating or dissolving an alkali metal alkoxide in an alcohol; dissolving piperine and/or a metabolite or derivative thereof in said alkali metal alkoxide alcohol solution; and drying said solution under anhydrous conditions. In certain embodiments, the alcohol is ethanol and the alkali metal alkoxide is an alkali metal ethoxide. In certain embodiments, the alkali metal alkoxide in an alcohol solution is obtained by dissolving an alkali metal ethoxide in an alcohol or by adding an alkali metal to an ethanol solution. In certain embodiments, the drying takes place in low atmospheric humidity conditions. In certain embodiments, the drying takes place at atmospheric humidity below 30%. In certain embodiments, the drying takes place at atmospheric humidity below 20%. In certain embodiments, the drying takes place at atmospheric humidity below 10%. In certain embodiments, the drying takes place in a vacuum. In certain embodiments, the anhydrous conditions consist of a water content below 1% in either gaseous or liquid compositions.

In even another aspect, the method of treating a piperine improvable ailment, disease, or condition is disclosed comprising administering to a subject the composition of any embodiment herein. In certain embodiments, the piperine improvable ailment, disease, or condition is one of allergic rhinitis (hay fever), asthma, depression, diarrhea, dysmenorrhea, dyspepsia, fall prevention, fatigue, flatulence, obesity, pain (either chronic or acute), rhinosinusitis, lack of sexual desire, vitiligo, or smoking cessation. In certain embodiments, the subject is administered between 1-5000 mg of the composition. In certain embodiments, the subject is administered between 1-1000 mg of the composition.

In another aspect, a method of improving athletic performance is disclosed comprising administering to a subject 1-5000 mg of the composition of any embodiment herein. In certain embodiments, the subject is administered between 1-1000 mg of the composition.

In still another aspect, a method of improving the bioavailability and/or effectiveness of a dietary supplement, nutraceutical, or pharmaceutical is disclosed comprising co-administering said supplement, nutraceutical, or pharmaceutical with the composition of any embodiment herein. In certain embodiments, the improvement of bioavailability and/or effectiveness of said dietary supplement, pharmaceutical, or nutraceutical is greater as compared to co-administering an equal piperine dose as in the complex of piperine and/or a metabolite or derivative thereof and an alkali metal alkoxide of an alkali metal. In certain embodiments, the subject is administered between 1-1000 mg of the composition.

The foregoing and other aspects, features, and advantages will be apparent from the DESCRIPTION and DRAWINGS, and from the CLAIMS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will hereinafter be described in conjunction with the appended and/or included DRAWINGS, where like designations denote like elements.

FIG. 1—A photograph depicting an orange solution of methanol, lithium, and piperine.

FIG. 2—A photograph of a deep yellow powder obtained by drying out the solution of FIG. 1 under vacuum at 50° C.

FIG. 3—Fourier Transform Infrared (FTIR) spectroscopy of the piperine-lithium methoxide complex.

FIG. 4—Fourier Transform Infrared (FTIR) spectroscopy of the piperine.

FIG. 5—A photograph of a yellowish powder of dried piperine-Lithium Propoxide complex.

FIG. 6—Fourier Transform Infrared (FTIR) spectroscopy of the piperine-Lithium Propoxide complex.

FIG. 7—A photograph of a yellow powder of dried piperine Lithium ethoxide complex.

FIG. 8—A photograph of piperine Lithium ethoxide complex dissolved in 1000 ml of water providing a yellow solution.

FIG. 9—Fourier Transform Infrared (FTIR) spectroscopy of the piperine Lithium ethoxide complex.

FIG. 10—Photographs showing solubilization of piperine-sodium ethoxide complex.

FIG. 11—Fourier Transform Infrared (FTIR) spectroscopy of the piperine-sodium ethoxide complex.

FIG. 12—Charts showing absorption of indicated amino acids after ingesting 30 grams of whey protein in 500 ml water (10/24/2024), 30 grams of whey protein in 500 ml water and 150 mg piperine-lithium ethoxide complex (10/29/2024), and 30 grams of whey protein in 500 ml water and 500 mg of piperine (12/5/2024).

The figures herein are for illustrative purposes only.

### DESCRIPTION

Detailed aspects and applications of the disclosure are described below in the following detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous very specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

### Overview

The present disclosure relates to a water-soluble and/or dispersible piperine and methods of producing the water-soluble and/or dispersible piperine. In example embodiments, the piperine is complexed with an alkoxide. Non-limiting alkoxides include lithium methoxide, lithium ethoxide, lithium propoxide, lithium isopropoxide, lithium butoxide, lithium isobutoxide sodium methoxide, sodium ethoxide, sodium propoxide, sodium isopropoxide, sodium butoxide, potassium methoxide, potassium ethoxide, potassium propoxide, potassium isopropoxide, potassium butoxide, potassium isobutoxide and the like. In example embodiments, water-soluble and dispersible piperine complexes can be used in a variety of pharmaceutical, nutraceutical, dietary supplement and cosmetic applications.

### Terminology and Definitions

The technical terms and expressions used within the scope of this application are generally to be given the meaning commonly applied to them in the pertinent art of pharmaceutical and nutraceutical formulations. All of the following term definitions apply to the complete content of this application.

The word “comprising” does not exclude other elements or steps, and the indefinite article “a” or “an” does not exclude a plurality. A single step may fulfil the functions of several features recited in the claims. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural referents unless the context clearly dictates otherwise. Thus, for example, reference to “a step” includes reference to one or more of such steps.

The term “and/or” means (a) or (b) or both (a) and (b).

The terms “about”, “essentially” and “approximately” in the context of a given numerate value or range refers to a value or range that is within 20%, within 10%, or within 5%, 4%, 3%, 2% or 1% of the given value or range.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. The recitation of numerical ranges by endpoints includes all numbers and fractions subsumed within the respective ranges, as well as the recited endpoints.

The term “isolated” refers to any entity that is taken from its natural milieu, but the term does not connote any degree of purification.

The term “food” should refer to any nutritious substance that people or animals eat or drink or that plants absorb in order to maintain life and growth.

The term “dietary supplement” shall refer to products that are intended to add to or supplement the diet and are different from conventional food. Dietary supplements come in a variety of forms, including tablets, capsules, gummies, and powders as well as drinks and energy bars to name a few.

The term nutraceutical refers to a food or dietary ingredient that has health benefits and/or medicinal properties.

The term “elemental metal” refers to the uncharged, metallic form of a metal, and not other charged forms such as oxides, hydroxides, and salts.

The term “metallic metal” will refer to any of the following metals in their metallic (uncharged) form: magnesium, calcium, lithium, zinc, sodium, potassium, beryllium, rubidium, cesium, aluminum, gallium, indium, tin, bismuth, scandium, titanium, vanadium, chromium, manganese, cobalt, manganese, scandium, titanium, nickel, copper, zinc, yttrium, zirconium, niobium, molybdenum, technetium, ruthenium, rhodium, palladium, silver, cadmium, lanthanum, hafnium, tantalum, tungsten, rhenium, osmium, iridium, platinum, gold, and manganese. In some implementations, “metallic metal” will not include metals in their charged forms, such as oxides, hydroxides, salts and the like.

The term “hydride” shall refer to a binary compound formed by hydrogen and another, usually more electropositive, metallic metal, such as sodium hydride (NaH) or Magnesium hydride (MgH₂) or Lithium hydride (LiH) or Potassium hydride (KH) and hydrides of any of the following metals: magnesium, calcium, lithium, zinc, beryllium, rubidium, cesium, aluminum, gallium, indium, tin, bismuth, scandium, titanium, vanadium, chromium, manganese, cobalt, manganese, scandium, titanium, nickel, copper, zinc, yttrium, zirconium, niobium, molybdenum, technetium, ruthenium, rhodium, palladium, silver, cadmium, lanthanum, hafnium, tantalum, tungsten, rhenium, osmium, iridium, platinum, gold, and manganese.

The term “hydroxide” shall refer to a diatomic anion with chemical formula OH— or shall refer to inorganic compounds which contain hydroxy groups (e.g., lithium hydroxide (LiOH)).

The term “piperine” refers to the following chemical compound: (2E,4E)-5-(2H-1,3-benzodioxol-5-yl)-1-(piperidin-1-yl) penta-2,4-dien-1-one (CAS #94-62-2). Piperine is an active component of Piper nigrum (commonly referred as black pepper). Despite the notation it is also present in white and red peppercorns of the same plant. It is a potent irritant for mammals, including humans, and produces a sensation of burning in any tissue with which it comes into contact. Secondary metabolites are compounds that are not directly involved in the primary metabolic processes essential for plant growth and development (such as photosynthesis or respiration) but play crucial roles in the plant's interaction with its environment. Piperine serves as a deterrent against herbivores and pests due to its pungent taste. This helps protect the plant from being consumed. It also exhibits antimicrobial properties, aiding the plant in defending against bacterial and fungal pathogens.

The term “pharmaceutically acceptable additive” or “additive” are terms used in their broadest sense. Particular implementations of the compositions described in this document may also comprise an additive (e.g. one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically effective additives from the disclosure in this document. In particular implementations, pharmaceutically acceptable additives may include, by non-limiting example, calcium phosphate, cellulose, stearic acid, crosscarmelose cellulose, magnesium stearate, and silicon dioxide.

Then term “pharmaceutically effective” is a phrase used in its broadest sense, including, by non-limiting example, effective in a clinical trial or for a specific patient. When used in a method claim, pharmaceutically effective will mean in a dose enough to achieve the claim's preamble.

Then term “pharmaceutically acceptable” is a phrase used in its broadest sense and may describe ingredients of a pharmaceutical composition that meet Food and Drug Administration (FDA) standards, United States Pharmacopeial Standards (USP), US Department of Agriculture (USDA) standards for food-grade materials, commonly accepted standards of the nutritional supplement industry, industry standards, botanical standards, or standards established by any individual. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

The term “disease” refers to a state of health of an animal (including humans) wherein the animal cannot maintain homeostasis, and wherein if the disease is not ameliorated then the animal's health continues to deteriorate.

In contrast, the term “disorder” in an animal refers to a state of health in which the animal (including humans) is able to maintain homeostasis, but in which the animal's state of health is less favorable than it would be in the absence of the disorder. Left untreated, a disorder does not necessarily cause a further decrease in the animal's state of health.

The term “ailment” may refer to any of “disease”, “condition”, or “disorder”.

A disease or disorder is “alleviated” if the severity of a symptom of the disease or disorder, the frequency with which such a symptom is experienced by a patient, or both, is reduced.

A disease is “cured” if the disease no longer shows any symptoms and appropriate diagnostic tests show absence of it on a patient.

As used herein, “treat,” “treatment,” and “treating,” a disease or disorder means reducing the severity and/or frequency, with which a sign and/or symptom of the disease or disorder is experienced by a subject. The subject may be symptomatic or asymptomatic at the time of treatment. In other words, “treat,” “treatment,” and “treating,” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

### Water-Soluble and Dispersible Piperine Compositions

Besides its culinary uses, piperine finds wide use in the nutraceutical supplement and drug industries to increase the absorption of bioactive compounds. Research indicates that piperine possesses several pharmacological properties, including antioxidant, anti-inflammatory, and anticancer activities. Piperine has been studied for its potential to aid in weight loss, improve digestion, and support cognitive function. Unfortunately, most of those properties manifest either in vitro or when piperine is directly injected to a subject, due to its low oral bioavailability.

Current research efforts to enhance the oral bioavailability of solid piperine include development of crystal polymorphs, salt formation with saccharin, self-emulsifying drug delivery system (SEDDS) and micro/nanoemulsions, complexion with cyclodextrins, polymeric nanocarriers, inorganic nanoparticles and hybrid nanoparticles and surfactants. In this last decade, various formulation strategies have been developed in order to overcome piperine's problems. However, among these formulations, thermodynamic stability is an issue, as piperine's metastable or amorphous state formation contributes to solubility and dissolution rate enhancement, leading to uncontrolled recrystallisation during storage. Metastable polymorphs tend to change to a more thermodynamically stable form in a relatively short time, hence close monitoring of polymorphic transformations is required during their formulation, manufacture and storage. In addition, polymorphs are often clinical failures once they hit the market, due to their transformation into undesirable forms that have lower solubility or are even clinically harmful. Amorphous recrystallisation brings back piperine's natural poor solubility and make the efforts made to manage it fruitless. Thus, more studies are required of formulations' crystallization behaviors, such as nucleation induction time, supersaturation tests, and shelf stability (the above-discussed studies have not done much to predict recrystallisation in storage). Solubility enhancement has been demonstrated with solubilizers such as cyclodextrins, but studies have reported that these solubilization mechanisms may forfeit intestinal absorption and lower apparent permeability due to reduced free drug availability for absorption. Therefore, there is a long-felt need in the art for an easy to produce, stable and effective water soluble form of piperine. The inventors have solved this problem.

### Biosynthesis of Piperine

Piperine is synthesized through the phenylpropanoid pathway, a common route for the production of various secondary metabolites in plants. The process involves the combination of piperidine (a derivative from the amino acid lysine) and piperic acid (derived from phenylalanine) to form piperine. Extraction of piperine has been described (see, e.g., K. V. Padalkar & V. G. Gaikar (2008) Extraction of Piperine from Piper Nigrum (Black Pepper) by Aqueous Solutions of Surfactant and Surfactant+Hydrotrope Mixtures, Separation Science and Technology, 43:11-12, 3097-3118; and S Bahri et al 2019 J. Phys.: Conf. Ser. 1338 012010). Various synthetic routes to make piperine have also been described in the literature (see, e.g., Tiwari A, Mahadik K R, Gabhe S Y. Piperine: A comprehensive review of methods of isolation, purification, and biological properties. Med Drug Discov. 2020; 7:100027).

To clarify Piperine Isomers and Piperine Metabolites/Derivatives, it is usually understood that “geometric isomers of piperine” include the naturally occurring and/or photoisomerization-related isomers chavicine, isochavicine, and isopiperine, in addition to piperine itself. As used herein, a “piperine metabolite or derivative” refers to a compound obtained from piperine by metabolic transformation or chemical modification, including, by way of example, hydrolysis products such as piperic acid (and salts and esters thereof), and piperine salts and esters.

In some embodiments, the alkoxide complexation methods described herein are carried out using piperine, which can be isolated from plants such as peppers or synthetically produced. In other embodiments, the same complexation methods may be carried out using one or more geometric isomers of piperine and/or one or more piperine metabolites/derivatives as defined above, using analogous anhydrous processing (e.g., contacting the selected compound with an alkali metal alkoxide in an anhydrous polar solvent and removing solvent under anhydrous conditions) to form a solid complex and/or salt/ion-pair that is water-dispersible and/or water-soluble.

The term “piperinoids” refers to a well-documented and understood category of compounds that are chemically similar to piperine and produce similar biological effects, such as the activation of the vanilloid type 1 (TRPV1) receptor. Their general formula is:

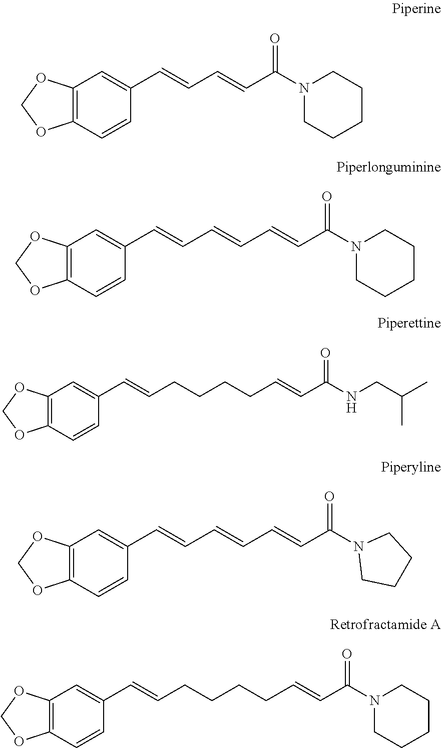

Some of them occur in nature while others are or can be made synthetically.

Complexes of Piperine with Alkoxides of Metals

The inventors have solved the problem of producing a stable and effective water soluble form of piperine by preparing novel complexes of piperine with alkoxides of metals such as lithium methoxide, lithium ethoxide, lithium propoxide, lithium butoxide, sodium methoxide, sodium ethoxide, sodium propoxide, sodium butoxide, potassium methoxide, potassium ethoxide, potassium propoxide, potassium butoxide and the like. Compounds produced by the methods described herein shall be referred to as alkoxide-piperinoid complexes. It will be understood that for purposes of this invention the order of the metal and pipercapsaicinoid in the naming of the complex is immaterial. For example, “sodium ethoxide-piperine complex” and “piperine-sodium ethoxide complex” refer to the same complexes. Also, unless otherwise specified, the term metal shall refer to one of sodium, lithium or potassium, in its metallic, uncharged state.

In example embodiments, the preparation method consists of first dissolving in an anhydrous polar solvent, preferably an alcohol, such as methanol, ethanol, propanol, butanol, isopropyl alcohol, and/or another organic solvent such as dichloromethane, dimethyl sulfoxide (DMSO) an amount of an alkali metal such as Na, K and Li thus forming the ethoxide in situ, dissolving an appropriate amount of piperine in the alcohol and drying said solution in anhydrous conditions, such as in vacuum. However readily prepared ethoxide can be used to be dissolved in the alcohol, alongside the piperine and then dried out in anhydrous conditions. Another method would be dissolution of a hydride or hydroxide of a metal in an alcohol (e.g., ethanol) and then removal of water by a dehydrating agent that can be filtered out, such as CaO or a molecular sieve. In example embodiments, the weight ratio of piperine to the alkoxide is preferably 1:1 but lower/higher ratios of the alkoxide such as 0.1:1-1:1000, preferably 1:1-1:100 and more preferably 1:1-1:10 may be used. Using lower amounts of alkoxide than 0.1:1 can result in partial formation of the soluble piperine-base complex, while the rest will remain insoluble. The complexing of piperine with said alkoxides results in a crystalline waxy substance of yellowish/orange color. The compounds show excellent stability if protected from moisture and result in solubility much higher than that of piperine. Those results are highly surprising since piperine is an alkaloid (i.e. a compound whose solutions have an alkaline pH). In certain embodiments, the water-soluble and/or dispersible piperine complexes described herein provide enhanced aqueous dispersibility without requiring surfactants, emulsifiers, or water-soluble polymers used to physically solubilize piperine. As is well known in the art, such compounds de facto are less soluble in alkaline solutions-such as those produced by alkoxides dissolved in water. Even more surprisingly, testing showed no notable hydrolysis of piperine and piperidine despite the alkaline environment. Furthermore, the inventors have confirmed that addition of piperine to an alkaline solution (such as produced by an aqueous solution of an alkoxide) does not render piperine soluble.

It will be understood by the person of ordinary skill in the art that the above steps can be modified to reach the same results, for example two separate solutions of piperine and a metal alkoxide can be prepared, mixed and then evaporated, or a mixture of solvents instead of a single solvent can be used or even a mixture of different alkoxides can be used.

In example embodiments, drying of the solvent is preferably carried under vacuum or with dry air drying (i.e., drying with air with as little humidity content as possible), since the alkoxides are unstable at high levels of humidity. However, any conventional method for drying may be applied, such as oven drying, spray drying, rotary evaporation, microwave drying, desiccation and the like.

In example embodiments, the preferable solvent for the preparations of the invention is ethanol due to availability, ease to evaporate and relatively low toxicity. However, other appropriate polar solvents may be used, such as methanol, propanol, butanol, isopropyl alcohol, dichloromethane, dimethyl sulfoxide (DMSO) and the like.

In example embodiments, besides the hydroxides of the metals, for the purpose of the invention, the metals themselves after getting dissolved into an appropriate solvent can be used. For example, reacting potassium metal with ethanol will produce potassium ethoxide which solution can then be used to dissolve the piperinoids.

The disclosure also relates to compositions comprising metal ethoxides-piperinoid complexes or their derivatives and metabolites. In some implementations, the composition further comprises a dissolution enhancer, such as sodium carbonate for example. In some other implementations, the metal ethoxide-piperinoid complexes or their derivatives and/or metabolites are combined with a dietary ingredient, a food additive, and/or a pharmaceutically acceptable additive.

Supplements and ingredients that may be used in compositions containing metal-piperinoid complexes or their derivatives and metabolites to enhance the effectiveness of the composition include but are not limited to: 1,3-DMAA, 1,3-DMBA, 1,4-Butanediol, 1,4-DMAA, 1-Androsterone, 1-Epiandrosterone, 19-nor-DHEA,4-Androsterone, 5-decazol, 5-HTP, 5aOHP, 6-Bromo, 7,8-benzoflavone, 7-alpha-hydroxy-DHEA, 7-beta-hydroxy-DHEA, 7-Keto-DHEA, 7-Methoxyflavone, Abscess Root, Abuta, Acacia rigidula, Acai, Acerola, Acetyl-L-Carnitine, Ackee, Aconite, Activated Charcoal, Active hexose correlated compound (AHCC), Adenosine, Adrafinil, Adrenal extract, Adrue, Aegeline, African Wild Potato, Agar, Agaricus Mushroom, Agave, Agmatine, Agrimony, Ajuga nipponensis, Alanine, Albizia julibrissin, Alchemilla, Alder Buckthorn, Aletris, Alfalfa, Algal Oil, Algin, Alkanna, Allspice, Aloe, Alpha-GPC, Alpha-Ketoglutarate (AKG), Alpha-Linolenic Acid (ALA), Alpha-Lipoic Acid, Alpine Ragwort, Alpinia, Amaranth, Ambrette, American Adder's Tongue, American Chestnut, American Dogwood, American Elder, American Ginseng, American Hellebore, American Ivy, American Mistletoe, American Pawpaw, American Spikenard, American White Water Lily, Andarine, Andiroba, Andrographis, Androstenediol, Androstenedione, Androstenetrione, Androsterone, Angel's Trumpet, Angelica archangelica, Angostura, Anhydrous Crystalline Maltose, Anise, Annatto, Antineoplastons, Antioxidants, Apoaequorin, Apple, Apple Cider Vinegar, Apple Polyphenols, Apricot, Apricot Kernel, Arabinoxylan, Arenaria Rubra, Arimistane, Aristolochia, Arnica, Arrach, Arrowroot, Arsenic, Artemisia Herba-Alba, Artichoke, Arum, Asafoetida, Asarabacca, Ascophyllum nodosum, Ascorbigen, Ash, Ashitaba, Ashwagandha, Asian Water Plantain, Asparagus, Asparagus Racemosus, Aspartic Acid, Aspen, Astaxanthin, Astragalus, Atlantic Cedar, Atractylodes, Autumn Crocus, Avens, Avocado, Avocado soy unsaponifiables (ASU), Avocado Sugar Extract, Ayahuasca, Babassu, Bach Flower Remedies, Bacillus Coagulans, Bacopa, Bael, Baikal Skullcap, Ba Ji Tian, Bamboo, Banaba, Banana, Baobab, Barley, Basil, Bay Leaf, Bayberry, Bear's Garlic, Bee Pollen, Bee Venom, Beer, Beeswax, Beet, Belladonna, Benfotiamine, Benzoin, Berberine, Bergamot, Beta-Alanine, Beta-Carotene, Beta-Cryptoxanthin, Beta-Glucans, Beta-Hydroxybutyrate (BHB), Beta-Methylphenethylamine (BMPEA), Beta-Sitosterol, Betaine Anhydrous, Betaine Hydrochloride, Betel Nut, Beth root, Betony, Bifidobacterium animalis subsp. lactis, Bifidobacterium bifidum, Bifidobacterium breve, Bifidobacterium longum, Bilberry, Biotin, Birch, Bishop's Weed, Bismuth, Bismuth Nitrate, Bistort, Bitter Almond, Bitter Melon, Bitter Milkwort, Bitter Orange, Bitter Yam, Bittersweet Nightshade, Black Alder, Black Bryony, Black Cohosh, Black Currant, Black Haw, Black Hellebore, Black Hoof Mushroom, Black Horehound, Black Mulberry, Black Mustard, Black Nightshade, Black Pepper, Black Psyllium, Black Raspberry, Black Rice, Black Root, Black Seed, Black Tea, Black Walnut, Blackberry, Blackthorn, Blessed Thistle, Blond Psyllium, Bloodroot, Blue Cohosh, Blue Flag, Blue-Green Algae, Blueberry, Bog Bilberry, Bog Labrador Tea, Bogbean, Bois de Rose Oil, Boldo, Boneset, Borage, Boron, Boswellia Serrata, Bovine Cartilage, Bovine Colostrum, Boxwood, Branched-chain Amino Acids (BCAA), Breadfruit, Brewer's Yeast, Brickellia, Bridelia, Broccoli, Broccoli Sprout, Bromelain, Brown Rice, Brussels Sprout, Bryonia, Buchu, Buck's-horn Plantain, Buckhorn Plantain, Buckwheat, Bugle, Bugleweed, Bulbine natalensis, Bulbous Buttercup, Bupleurum, Burdock, Burning Bush, Burr Marigold, Butcher's Broom, Butea Superba, Butterbur, Buttercup, Butternut, Butylated hydroxytoluene (BHT), Cabbage, Cade, Caffeic Acid, Caffeine, Cajeput Oil, Calabar Bean, Calabash Chalk, Calamint, Calamus, Calanus Oil, Calcium, Calcium D-Glucarate, Calea Zacatechichi, Calendula, California Poppy, Calotropis, Calumba, Camphor, Camu Camu, Canada Balsam, Canadian Fleabane, Canadian Hemp, Canaigre, Cananga Oil, Cannabichromene (CBC), Cannabidiol (CBD), Cannabidivarin (CBDV), Cannabigerol (CBG), Cannabinol (CBN), Cannabis, Canola Oil, Canthaxanthin, Capers, Caprylic Acid, Capsicum, Caralluma, Caraway, Carbon 60 (C60), Cardamom, Cardarine, Carlina, Carnosine, Carob, Carqueja, Carrageenan, Carrot, Cascara Sagrada, Cascarilla, Casein Peptides, Casein Protein, Cashew, Cassava, Cassia Auriculata, Cassia Cinnamon, Cassia Nomame, Cassie Absolute, Castor Bean, Castoreum, Cat's Claw, Cat's Foot, Catechu, Catnip, Catuaba, Cauliflower, Celery Centaury, Cereus, Cesium, Cetylated Fatty Acids (CFAs), Ceylon Cinnamon, Ceylon Leadwort, Cha de Bugre, Chaga, Chanca Piedra, Chaparral, Chaulmoogra, Cheken, Chelation Therapy Products, Chenopodium Oil, Chervil, Chia, Chickweed, Chicory, Chinese Cucumber, Chinese Mallow, Chinese Prickly Ash, Chirata, Chitosan, Chive, Chlorella, Chlorine Dioxide, Chlorophyll, Chlorophyllin, Chokeberry, Choline, Chondroitin Sulfate, Chromium, Chrysanthemum, Chrysin, Chuchuhuasi, Chymotrypsin, Cilantro, Cinchona, Cissus Quadrangularis, Cistanche deserticola, Citicoline, Citric Acid, Citronella oil, Clary Sage, Clay, Clematis recta, Clivers, Clove, Clown's Mustard plant, Clubmoss, Cnidium, Cobalt, Coca, Cocillana, Cocoa, Coconut, Coconut Oil, Coconut Water, Cod Liver Oil, Codonopsis, Coenzyme Q10, Coffee, Coffee Charcoal, Cola Nut, Coleus, Collagen Peptides, Collagen Type I (native), Collagen Type II (native), Collard, Colloidal Minerals, Colloidal Silver, Colocynth, Coltsfoot, Columbine, Combretum micranthum, Comfrey, Common Stonecrop, Condurango, conjugated Linoleic Acid (CLA), Copaiba Balsam, Copper, Coral, Cordyceps, Coriander, Corkwood Tree, Corn Poppy, Corn Silk, Cornflower, Corydalis yanhusuo, Costus, Cotton, Couch Grass, Cowhage, Cowslip, Cramp bark, Cranberry, Creatine, Croton Seeds, Cubebs, Cucumber, Cudweed, Cumin, Cursed Buttercup, Cyanostane, Cyclamen, Cypress, D-Mannose, Daffodil, Damiana, Dandelion, Danshen, Date Palm, Datura Wrightii, Deanol, Deer Velvet, Delta-8-Tetrahydrocannabinol (Delta-8-THC), Delta-9-Tetrahydrocannabinol (THC), Dendrobium, Desert Parsley, Devil's Claw, Devil's Club, DHEA, Diacylglycerol, Diatomaceous earth, Diindolylmethane, Diiodothyronine, Dill, Dimethylglycine (DMG), Dimethylhexylamine (DMHA), Dimethylsulfoxide (DMSO), Diosmin, Divi-Divi, Docosahexaenoic Acid (DHA), Dodder, Dolomite, Dong Quai, Douglas fir, Dragon Fruit, Dragon's Blood, Duckweed, Dulse, Durabolin, Durian, Dusty Miller, Dwarf Elder, Dwarf Pine Needle, Dyer's Broom, Dymethazine, Eastern Hemlock, Eastern Red Cedar, Ecdysteroids, Echinacea, Ecklonia Cava, Eicosapentaenoic Acid (EPA), Elderberry, Elderflower, Elecampane, Elemi, Eleuthero, Ellagic Acid, Elm Bark, Emu Oil, English Adder's Tongue, English Horsemint, English Ivy, English Walnut, Ephedra, Epiandrosterone, Epistane, Equol, Ergot, Ergothioneine, Eryngo, Eucalyptus, Euphorbia cyparissias, Euphorbia hirta, European Barberry, European Buckthorn, European Chestnut, European Five-finger Grass, European Mandrake, European Mistletoe, Eurycoma Longifolia, Evening Primrose, Evodia, Eyebright, Fadogia Agrestis, False Unicorn, Fennel, Fenugreek, Fermented Milk, Fermented Wheat Germ Extract, Fever Bark, Feverfew, Ficin, Field Scabious, Fig, Figwort, Fir, Fireweed, Fish Oil, Flaxseed, Flaxseed Oil, Fluoride, Fly Agaric Mushroom, Fo-Ti, Folic Acid, Fool's Parsley, Forget-Me-Not, Forsythia, Foxglove, Frankincense, Fringetree, Frostwort, Fructo-Oligosaccharides (FOS), Fucus Vesiculosus, Fulvic Acid, Fumitory, G,Galacto-oligosaccharides (GOS), Galbanum, Galphimia Glauca, Gamboge, Gamma Butyrolactone (GBL), Gamma-Aminobutyric Acid (GABA), Gamma-Hydroxybutyrate (GHB), Gamma-Linolenic Acid (GLA), Gamma-oryzanol, Garcinia, Garden Cress, Gardenia, Garlic, Gelatin, Gelsemium, Genistein Combined Polysaccharide, Gentian, German Chamomile, German Ipecac, German Sarsaparilla, Germander, Germanium, Ginger, Ginkgo, Globe Flower, Globemallow, Glossy Privet, Glucomannan, Glucosamine, Glucuronolactone, Glutaurine, Glutamine, Glutathione, Glycerol, Glycine, Glycolic Acid, Glycomacropeptide, Glyconutrients, Goa Powder, Goat's Rue, Goji, Golden Ragwort, Goldenrod, Goldenseal, Goldthread, Gossypol, Gotu Kola, Goutweed, Grains of Paradise, Grape, Grapefruit, Gravel Root, Graviola, Great Plantain, Greater Burnet, Greater Celandine, Greek Sage, Green Coffee, Green Tea, Griffonia Simplicifolia, Ground Ivy, Ground Pine, Groundsel, Guaiac Wood, Guar Gum, Guarana, Guarumo, Guava, Guggul, Gum arabic, Gumweed, Gymnema, Halodrol-50, Haronga, Hartstongue, Hawaiian baby woodrose, Hawthorn, Hazelnut, Heart's Ease, Heather, Hedge Mustard, Hedge-Hyssop, Hemlock, Hemlock Water Dropwort, Hemp, Hemp Agrimony, Hempnettle, Henbane, Henna, Herb Paris, Herb Robert, Hercules Club, Hesperidin, Hexadrone, Hexylamine, Hibiscus sabdariffa, Higenamine, Histidine, Holly, Hollyhock, Holy Basil, Homotaurine, Honey, Honeysuckle, Hoodia, Hops, Hordenine, Horny Goat Weed, Horse Chestnut, Horsemint, Horseradish, Horsetail, Hound's Tongue, Houseleek, Hu Zhang, Humic Acid, Huperzine A, Hyacinth Bean, Hyaluronic Acid, Hydrangea, Hydrazine Sulfate, Hydroxymethylbutyrate (HMB), Hyperimmune Egg, Hyssop, Iboga, Iceland Moss, Idebenone, Ignatius Bean, Immortelle, Indian Cassia, Indian Gooseberry, Indian Long Pepper, Indian Snakeroot, Indigo pulchra, Indium, Indole-3-Carbinol, Inosine, Inositol, Inositol Nicotinate, Inulin, Iodine, IP-6, Ipecac, Iporuru, Ipriflavone, Iron, Irvingia gabonensis, Isatis, Isopropylnorsynephrine, Ivy Gourd, Jaborandi, Jackfruit, Jalap, Jamaican Dogwood, Jambolan, Japanese Apricot, Japanese Mint, Japanese Persimmon, Jasmine, Java Tea, Javanese Turmeric, Jenny Craig Diet, Jequirity, Jewelweed, Jiaogulan, Jimson Weed, Job's Tears, Jojoba, Juniper, Justicia pectoralis, K2/Spice, Kale, Kamala, Kaolin, Karaya Gum, Kava, Kefir, Ketogenic diet, Khat, Khella, Kinetin, Kiwi, Knotweed, Kohlrabi, Kombucha, Korean Pine, Kousso, Kratom, Krill Oil, Kudzu, L-Arginine, L-Carnitine, L-Citrulline, L-Cysteine, L-Ornithine-L-Aspartate, L-Tryptophan, Labdanum, Laburnum, Lactase, Lactic Acid, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus crispatus, Lactobacillus delbrueckii, Lactobacillus gasseri, Lactobacillus helveticus, Lactobacillus jensenii, Lactobacillus johnsonii, Lactoferrin, Lady's Bedstraw, Laminaria, Larch Arabinogalactan, Larch Turpentine, Lathyrus, Latilactobacillus sakei, Laurelwood, Lauric Acid, Lavender, Lavender Cotton, Laxogenin, Lecithin, Lemon, Lemon Balm, Lemon Eucalyptus, Lemon Verbena, Lemongrass, Lentinan, Lesser Celandine, Levant Berry, Levilactobacillus brevis, Licorice, Ligandrol, Ligilactobacillus salivarius, Lily-of-the-Valley, Lime, Limonene, Limosilactobacillus fermentum, Limosilactobacillus reuteri, Linden, Lingonberry, Lion's Mane Mushroom, Lipase, Lithium, Liver Extract, Liverwort, Lobelia, Logwood, Lorenzo's Oil, Lotus, Lousewort, Lovage, Luffa, Lunasin, Lungmoss, Lungwort, Lupin, Lutein, Lychee, Lycopene, Lysine, M1-4ADD, Maca, Macadamia Nut, Mace, Madagascar Periwinkle, Madder, Magnesium, Magnolia, Maidenhair Fern, Maitake Mushroom, Malabar Nut, Male Fern, Malic Acid, Mallow, Manaca, Manchurian Thorn, Manganese, Mangosteen, Manna, Maqui, Maral Root, Maritime Pine, Marjoram, Marsh Blazing Star, Marsh Labrador Tea, Marsh Marigold, Marshmallow, Masterwort, Mastic, Meadowsweet, Medium Chain Triglycerides (MCTs), Melanotan, Melatonin, Mentabolan, Mercury Herb, Mesoglycan, Methasterone, Methionine, Methoxydienone, Methoxylated Flavones, Methyldiazinol, Methylstenbolone, Methylsulfonylmethane (MSM), Methylsynephrine, Mezereon, Milk Thistle, Miracle Fruit, Molybdenum, Moneywort, Monolaurin, Monterey Pine, Moringa, Mormon Tea, Motherwort, Mountain Ash, Mountain Flax, Mountain Laurel, Mouse-Ear Hawkweed, Mugwort, Muira Puama, Mullein, Musk, Myrcia, Myrrh, Myrtle, N, N-DMPEA, N-Acetyl Cysteine (NAC), N-Methyltyramine, NADH, Nasturtium, Nattokinase, Neem, Nerve Root, New Jersey Tea, New Zealand Green-Lipped Mussel, Niacin, Niacinamide, Nicotinamide Mononucleotide, Niauli oil, Nickel, Nicotinamide Riboside, Nikko Maple, Noni, Northern Prickly Ash, Norway Spruce, Nutmeg, Nux Vomica, Oak Moss, Oats, Octacosanol, Octopamine, Oleander, Oleic Acid, Olive, Olive Oil, Omega-6 Fatty Acids, Onion, Oolong Tea, Orchic extract, Oregano, Oregon Grape, Oriental Arborvitae, Ornamental Marigold, Ornithine, Ornithine Ketoglutarate (OKG), Orris, Oscillococcinum, Osha, Ostarine, Ostrich Fern, Oswego Tea, Ox-Eye Daisy, Padang Cassia, Pagoda Tree, Palm Oil, Palmitoylethanolamide (PEA), Panax Ginseng, Panax Notoginseng, Pancreatic Enzyme Products, Pangamic Acid, Pantethine, Pantothenic Acid, Pao Pereira, Papain, Papaya, Para-Aminobenzoic Acid (PABA), Pareira, Parsley, Parsley Piert, Parsnip, Partridgeberry, Passion Flower, Pata De Vaca, Patchouli Oil, Pau D'Arco, Pea Protein, Peanut Oil, Pear, Pectin, Pedunculate Oak, Pellitory, Pellitory-of-the-Wall, Pennyroyal, Peony, Peppermint, Perilla, Perillyl Alcohol, Periwinkle, Peru Balsam, Peyote, Phaseolus Vulgaris, Pheasant's Eye, Phellodendron, Phenethylamine (PEA), Phenibut, Phenpromethamine, Phenylalanine, Phlorizin, Phosphate Salts, Phosphatidylcholine, Phosphatidylserine, Phytase, Picamilon, Picrorhiza, Pimpinella, Pinellia Ternata, Pink Root, Pipsissewa, Piracetam, Pitcher Plant, Plant Sterols, Pleurisy root, Plum, Podophyllum, Poinsettia, Poison Ivy, Pokeweed, Polarity Therapy, Policosanol, Polydextrose, Polypodium Leucotomos, Pomegranate, Poplar, Poppy Seed, Poria Mushroom, Potassium, Potato, Potentilla, Pregnenolone, Premorse, Prickly Pear Cactus, Procaine, Progesterone, Proline, Propionyl-L-Carnitine, Propolis, Proteolytic Enzymes (Proteases), Psilocybin, Pu-erh Tea, Pulsatilla, Pumpkin, Purple Loosestrife, Purple Nut Sedge, Pygeum, Pyrethrum, Pyruvate, Quassia, Quebracho Blanco, Queen's Delight, Quercetin, Quillaia, Quince, Quinoa, Rabdosia Rubescens, Radish, Raspberry Ketone, Rauvolfia Vomitoria, Rauwolscine, Red Clover, Red Maple, Red Raspberry, Red Sandalwood, Red Soapwort, Red Yeast Rice, Red-spur Valerian, Reed Herb, Rehmannia, Reishi Mushroom, Resveratrol, Rhatany, Rhodiola, Rhubarb, Riboflavin, Ribose, Rice Bran, Rice Bran Arabinoxylan Compound, Rice protein, RNA and DNA, Rock Rose, Roman Chamomile, Rooibos, Rose Geranium Oil, Rose Hip, Rosemary, Royal Jelly, Rue, Rupturewort, Rusty-leaved Rhododendron, Rutin, Rye Grass, S-23, Saccharomyces Boulardii, Safed Musli, Safflower, Saffron, Sage, Saigon Cinnamon, Salacia, Salatrim, Salep, Salvia divinorum, SAMe, Samphire, Sandy Everlasting, Sangre de Grado, Sanicle, Sarsaparilla, Sassafras, Savin Tops, Saw Palmetto, Scarlet Pimpernel, Sceletium, Schisandra, Schizonepeta, Scopolia, Scotch Broom, Scotch Thistle, Scurvy Grass, Sea Buckthorn, Sea Moss, Secretin, Securinega Suffruticosa, Selenium, Self-Heal, Senega, Senna, Serine, Serrapeptase, Sesame, Sessile Oak, Shark Cartilage, Shark Liver Oil, Shea Butter, Shellac, Shepherd's Purse, Shiitake Mushroom, Siberian Cocklebur, Sida Cordifolia, Silicon, Simaruba, Sitostanol, Skullcap, Skunk Cabbage, Slippery Elm, Smartweed, Smooth Alder, Snake Skin, Sodium, Sodium Bicarbonate, Sodium Tetrachloroaurate, Solomon's Seal, Sorghum, Sorrel, Soy, Soybean Oil, Spanish Broom, Spanish Origanum Oil, Spearmint, Spinach, Spiny Restharrow, Spleen extract, Spotted Geranium, Squalamine, Squill, St. John's Wort, Star Anise, Star of Bethlehem, Stavesacre, Stenabolic, Stereospermum, Stevia, Stinging Nettle, Stone Root, Storax, Strawberry, Streptococcus thermophilus, Strontium, Strophanthus, Succinate, Sulbutiamine, Sulforaphane, Sulfur, Suma, Sumbul, Summer Savory, Sundew, Sunflower Oil, Superoxide Dismutase (SOD), Swallowroot, Swamp Milkweed, Sweet Almond, Sweet Annie, Sweet Cherry, Sweet Cicely, Sweet Clover, Sweet Gale, Sweet Orange, Sweet Sumac, Sweet Vernal Grass, Sweet Violet, Sweet Woodruff, Syrian Rue, Tamarind, Tamarix Dioica, Tangerine, Tannic acid, Tansy, Tansy Ragwort, Tapioca, Tarragon, Tart Cherry, Tartaric Acid, Taurine, Tea Tree Oil, Teazle, Terminalia, Testolone, Tetrahydrocannabivarin (THCV), Theacrine, Theaflavin, Theanine, Thiamine, Threonine, Thuja, Thunder God Vine, Thyme, Thymus extract, Thyroid Extract, Tianeptine, Timothy Grass, Tin, Tinospora Cordifolia, Tiratricol, Tocotrienols, Tolu Balsam, Tomato, Tonka Bean, Toothed Clubmoss, Tormentil, Tragacanth, Trailing Arbutus, Transfer Factor, Traveler's Joy, Tree of Heaven, Tree Tobacco, Tree Turmeric, Trendione, Tribulus, Trichopus Zeylanicus, Tronadora, Trypsin, Tung Seed, Turkey Corn, Turkey Tail Mushroom, Turmeric, Turpentine Oil, Turtlehead, Tylophora, Tyramine, Tyrosine, Ubiquinone, Umckaloabo, Usnea, Uva Ursi, Uzara, Valerian, Vanadium, Vanilla, Verbena, Veronica, Vetiver, Vietnamese Coriander, Vinpocetine, Vitamin A, Vitamin B1, Vitamin B2, Vitamin B12, Vitamin B6, Vitamin C, Vitamin D, Vitamin E, Vitamin K, Vitamin O, Wafer Ash, Wahoo, Wallflower, Wasabi, Water Avens, Water Dock, Water Hemlock, Watercress, Wheat Bran, Wheatgrass, Whey Protein, White Dead Nettle Flower, White Hellebore, White Horehound, White Lily, White Mulberry, White Mustard, White Oak, White Pepper, White Sandalwood, Wild Carrot, Wild Cherry, Wild Daisy, Wild Indigo, Wild Lettuce, Wild Mint, Wild Thyme, Wild Yam, Willard Water, Willow Bark, Wine, Winter Cherry, Winter Savory, Wintergreen, Witch Hazel, Wood Anemone, Wood Sage, Wood Sorrel, Wormseed, Wormwood, Xanthan Gum, Xanthoparmelia, Xylitol, Yarrow, Yellow Dock, Yellow Loosestrife, Yellow Toadflax, Yerba Mansa, Yerba Mate, Yerba Santa, Yew, Yin Chen, Ylang Ylang Oil, Yogurt, Yohimbe, Yucca, Zeaxanthin, Zedoary, Zinc, Zizyphus, carnitine or a salt, ester, or amide suitable for human ingestion thereof, acetyl-L-carnitine or a salt, ester, or amide suitable for human ingestion thereof, propionyl-L-carnitine or a salt, ester, or amide suitable for human ingestion thereof, alpha lipoic acid, alpha-GPC, aniracetam, piracetam, phenylpiracetam, apoaequorin, artichoke, ashwagandha, astaxanthin, Bacopa monnieri, cat's claw, catuaba, cannabidiol (CBD), Celastris paniculatus, centrophenoxine, citicoline, Clitoria ternatea, coluracetam, Convolvulus pluricaulis, ubiquinone (for example, Coenzyme Q10), creatine or a salt, ester, or amide suitable for human ingestion thereof, choline or a salt, ester, or amide suitable for human ingestion thereof, docosahexaenoic acid (DHA), dimethylethanolamine (DMAE), forskolin, Ginkgo biloba, ginseng, gotu kola, guarana, kanna, kava kava, kratom, L-glutamine or a salt, ester, or amide suitable for human ingestion thereof, L-phenylalanine or a salt, ester, or amide suitable for human ingestion thereof, L-theanine or a salt, ester, or amide suitable for human ingestion thereof, L-tryptophan or a salt, ester, or amide suitable for human ingestion thereof, lecithin or a salt, ester, or amide suitable for human ingestion thereof, lemon balm, lion's mane mushroom, magnolia, medium chain triglycerides, reduced nicotinamide adenine dinucleotide (NADH), nefiracetam, nicotine, arecoline, noopept, oatstraw, oxiracetam, passion flower, phenibut, phosphatidylcholine, phosphatidylserine, picamilon, pine bark extract, pramiracetam, pyrroloquinoline quinone (PQQ), pterostilbene, resveratrol, rosemary, Rhodiola rosea, S-adenosyl methionine (SAMe), schizandrol-A, St. John's wort, sulbutiamine, taurine or a salt, ester, or amide suitable for human ingestion thereof, turmeric, L-tyrosine or a salt, ester, or amide suitable for human ingestion thereof, N-acetyl-L-tyrosine or a salt, ester, or amide suitable for human ingestion thereof, uridine, valerian, vinpocetine, thiamine or a salt, ester, or amide a salt, ester, or amide suitable for human ingestion thereof, niacin or a salt, ester, or amide suitable for human ingestion thereof, pantothenic acid or a salt, ester, or amide suitable for human ingestion thereof, vitamin B6 or a salt, ester, or amide suitable for human ingestion thereof, vitamin B8 or a salt, ester, or amide suitable for human ingestion thereof, vitamin B9 or a salt, ester, or amide suitable for human ingestion thereof, vitamin B12 or a salt, ester, or amide suitable for human ingestion thereof, and yerba mate.

In example embodiments, compositions and/or formulations of the present disclosure may be administered in any form, including a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof for example. In example embodiments, compositions and/or formulations of the present disclosure may also include an acceptable additive (e.g., one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or an acceptable carrier (e.g., one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). In example other embodiments, the compositions may be used to readily prepare injectable formulations containing piperinoid-meta complex, by for example diluting them with water for injection.

Implementations of the metal-piperinoid complexes may conveniently be presented in unit dosage form. Unit dosage formulations may be those containing a daily dose or unit, a daily sub-dose, or an appropriate fraction thereof, of the administered components as described herein.

In example embodiments, a dosage unit may include the metal-piperinoid complexes or their derivatives and metabolites and at least one pharmaceutically acceptable additive. The additive may be, but not limited to, a sugar or a casein containing compound such as casein powder, to prevent gastrointestinal discomfort upon ingestion.

In example embodiments, the dosage for oral use of the metal-piperinoid complexes can be from 0.01 to 5000 mg per day, preferably, 0.01 to 1000 mg per day, split into one or more dosages. For exercise performance enhancing purposes, the formulation is administered (for example, by ingestion) prior to exercise, for example any time between 3 days prior to exercise and right before exercise. In certain implementations, the formulation is administered half an hour before exercise.

In example embodiments, the dosage units may be in a form suitable for administration by standard routes. In general, the dosage units may be administered, by non-limiting example, by the topical (including buccal and sublingual), transdermal, oral, rectal, ophthalmic (including intravitreal or intracameral), nasal, vaginal, and/or parenteral (including subcutaneous, intramuscular, intravenous, intradermal, intratracheal, and epidural) routes.

For the exemplary purposes of this disclosure, oral delivery may be a particularly advantageous delivery route for administration to humans and animals of implementations of a pharmaceutical composition, optionally, formulated with appropriate pharmaceutically acceptable additives to facilitate administration.

It should be appreciated that any of the components of particular implementations of the metal complexes of piperinoids may be used as supplied commercially, or may be preprocessed by, by non-limiting example, any of the methods and techniques of agglomeration, air suspension chilling, air suspension drying, balling, coacervation, comminution, compression, pelletization, cryopelletization, extrusion, granulation, homogenization, inclusion compoundation, lyophilization, melting, mixed, molding, pan coating, solvent dehydration, sonication, spheronization, spray chilling, spray congealing, spray drying, and/or other processes known in the art depending in part on the dosage form desired. The various components may also be pre-coated or encapsulated as known in the art. It will also be clear to one of ordinary skill in the art that appropriate additives may also be introduced to the composition or during the processes to facilitate the preparation of the dosage forms, depending on the need of the individual process.

Those of ordinary skill in the art will be able to readily select manufacturing equipment and pharmaceutically acceptable additives or inert ingredients to manufacture implementations of the metal-piperinoid complexes. For the exemplary purposes of this disclosure, some examples of pharmaceutically acceptable additives or inert ingredients and manufacturing process are included below, particularly those that relate to manufacture of implementations of the metal-piperinoid complexes in tablet form. Notwithstanding the specific examples given, it will be understood that those of ordinary skill in the art will readily appreciate how to manufacture implementations of the metal-piperinoid complexes according to the other methods of administration and delivery disclosed in this document.

A particular implementation of the metal-piperinoid complexes may include a lubricant. Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may comprise, for example, magnesium stearate, calcium stearate, talc, and colloidal silica.

Particular implementations of the metal alkoxide-piperinoid complexes may also include a binder. Binders are any agents used to impart cohesive qualities to powdered material through particle-particle bonding. Binders may include, for example, matrix binders (e.g., dry starch, dry sugars), film binders (e.g., celluloses, bentonite, sucrose), and chemical binders (e.g., polymeric cellulose derivatives, such as methyl cellulose, carboxy methyl cellulose, and hydroxy propyl cellulose); and other sugar, gelatin, non-cellulosic binders and the like.

Disintegrators may be used in particular implementations of the metal alkoxide-piperinoid complexes to facilitate the breakup or disintegration of tablets after administration. Disintegrators may include, for example, starch, starch derivatives, clays (e.g., bentonite), algins, gums (e.g., guar gum), cellulose, cellulose derivatives (e.g., methyl cellulose, carboxymethyl cellulose), croscarmellose sodium, croscarmellose cellulose, and other organic and inorganic materials.

Implementations of the metal alkoxide-piperinoid complexes may include diluents, or any inert substances added to increase the bulk of the metal-piperinoid complexes, such as potassium piperine complex to make a tablet a practical size for compression. Diluents may include, but are not limited to, for example, calcium phosphate, calcium sulfate, lactose, mannitol, magnesium stearate, potassium chloride, and citric acid, among other organic and inorganic materials.

Implementations of the metal alkoxide-piperinoid complexes may include buffering agents, such as sodium ethoxide and may be any one of an acid and a base, where the acid is, but not limited to, for example, propionic acid, p-toluenesulfonic acid, salicylic acid, stearic acid, succinic acid, tannic acid, tartaric acid, thioglycolic acid, or toluenesulfonic acid, and the base is, for example, ammonium hydroxide, potassium hydroxide, sodium hydroxide, sodium hydrogen carbonate, aluminum hydroxide, calcium carbonate, and other organic and inorganic chemicals.

Implementations of the metal alkoxide-piperinoid complexes or their derivatives and metabolites may also be administered through use of amphipathic lipid delivery systems (such as liposomes and unilamellar vesicles (A unilamellar liposome is a spherical liposome, a vesicle, bounded by a single bilayer of an amphiphilic lipid or a mixture of such lipids, containing aqueous solution inside the chamber.)), caplet systems, oral liquid systems, parenteral and/or intravenous systems, topical systems (creams, gels, transdermal patches, etc.), intranasal systems, rectal or vaginal systems, and many other delivery methods and/or systems known to those of ordinary skill in the art.

Those of ordinary skill in the art will readily be able to select additional pharmaceutically acceptable additives to enable delivery of implementations of a pharmaceutical composition from the disclosure in this document.

With respect to delivery of particular implementations of the metal alkoxide-piperinoid, for the exemplary purposes of this disclosure, tablets may be utilized. Tablets are any solid pharmaceutical dosage form containing a pharmaceutically acceptable active agent or agents to be administered with or without suitable pharmaceutically acceptable additives and prepared either by compression or molding methods well known in the art. Tablets have been in widespread use and remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be, for example, round, oval, oblong, cylindrical, rectangular, or triangular. The tablets may be optionally scored so that they may be separated into different dosages. Tablets may differ greatly in size and weight depending on the amount of the pharmaceutically acceptable active agent or agents present and the intended route of administration. Tablets are divided into two general classes: compressed tablets and molded tablets.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for, by non-limiting example, immediate release, pulsatile release, controlled release, extended release, delayed release, targeted release, synchronized release, or targeted delayed release. For release/absorption control, carriers may be made of various component types and levels or thicknesses of coats. Such diverse carriers may be blended in a dosage form to achieve a desired performance. In addition, the dosage form release profile may be affected by a polymeric matrix composition, a coated matrix composition, a multi-particulate composition, a coated multi-particulate composition, an ion-exchange resin-based composition, an osmosis-based composition, or a biodegradable polymeric composition.

While manufacture of implementations of the metal alkoxide-piperinoid complexes have been described in particular sequences of steps and/or in particular forms in the examples, it will be understood that such manufacture is not limited to the specific order of steps or forms as disclosed. Compounds of equivalent use/activity may be substituted for the compounds mentioned in the formulation. Any steps or sequences of steps of manufacture of implementations of the metal alkoxide-piperinoid complexes in any form are given as examples of possible steps or sequences of steps or potential forms and not as limitations, since many possible manufacturing processes and sequences of steps may be used to manufacture the metal-piperinoid complexes. For example, a combination of solvents or ethoxides or alcohols or metals could be used instead of one. In another example, a person could create a separate solution or piperine and another of the alkoxide and combine them.

### Methods of Treatment

As used herein, the term “disease” refers to a state of health of an animal (including humans) wherein the animal cannot maintain homeostasis, and wherein if the disease is not ameliorated then the animal's health continues to deteriorate. In contrast, as used herein, the term “disorder” in an animal refers to a state of health in which the animal (including humans) is able to maintain homeostasis, but in which the animal's state of health is less favorable than it would be in the absence of the disorder. Left untreated, a disorder does not necessarily cause a further decrease in the animal's state of health.

In other aspects, the disclosure relates to methods of treating a disease/condition/disorder (collectively called as ailments for simplicity) on a subject. Non-limiting diseases, disorders, and conditions include: allergic rhinitis (hay fever), asthma, depression, diarrhea, dysmenorrhea, dyspepsia, fall prevention, fatigue, flatulence, obesity, pain (either chronic or acute), rhinosinusitis, lack of sexual desire, vitiligo, and smoking cessation (effort to quit smoking cigarettes).

Besides the capacity of piperine to improve previously described “piperine improvable ailments”, piperine has been described as able to enhance aspects of a subject's living such as athletic performance that cannot be constituted as either a disease or a condition. A prime example is athletic performance (described as either endurance, speed, performance related to oxygen consumption, time to exhaustion or maximal strength on tests such as knee extension or 1 rep max (RM) bench press). In example embodiments, metal alkoxide-piperinoid complexes are used to increase athletic performance (e.g., in an otherwise healthy subject).

Further embodiments are illustrated in the following Examples which are given for illustrative purposes only and are not intended to limit the scope of the invention.

### EXAMPLES

### Example 1—Synthesis of Metal Alkoxide-Piperinoid Complexes

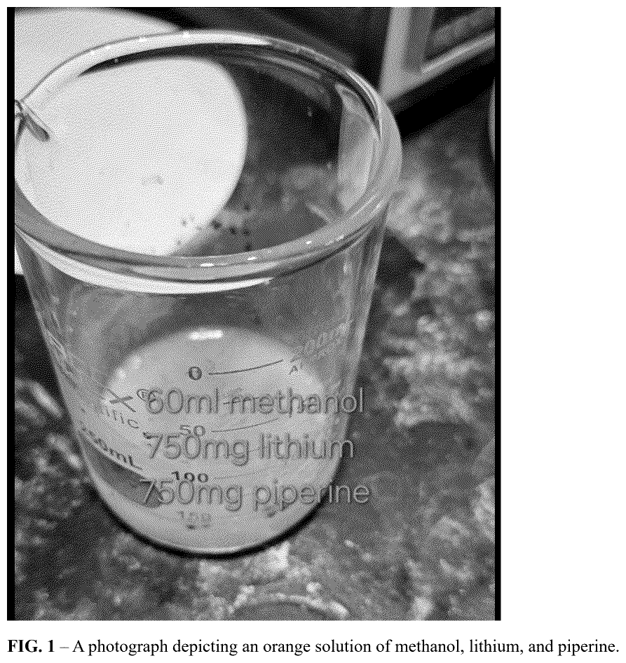

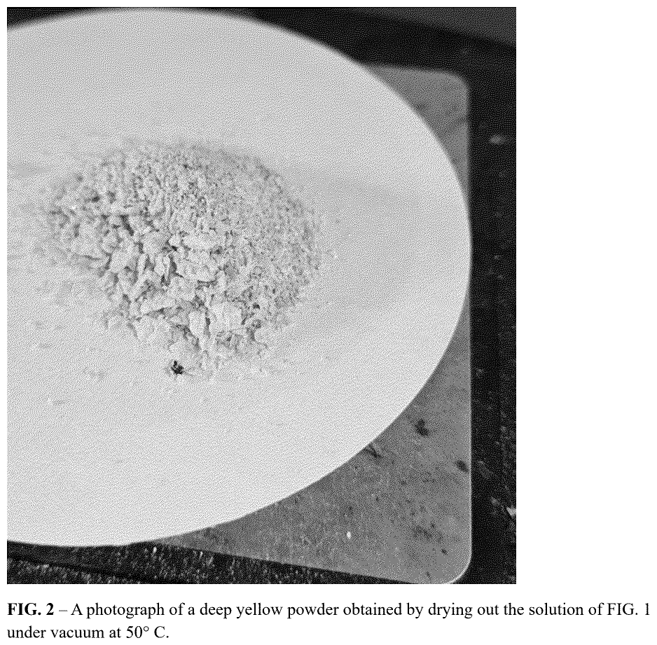

In 60 ml of absolute anhydrous methanol, 750 mg of metallic lithium was slowly dissolved. After the solution was left to cool down for a few minutes (the reaction is highly exothermic), 750 mg piperine was added. An orange solution was formed (FIG. 1).

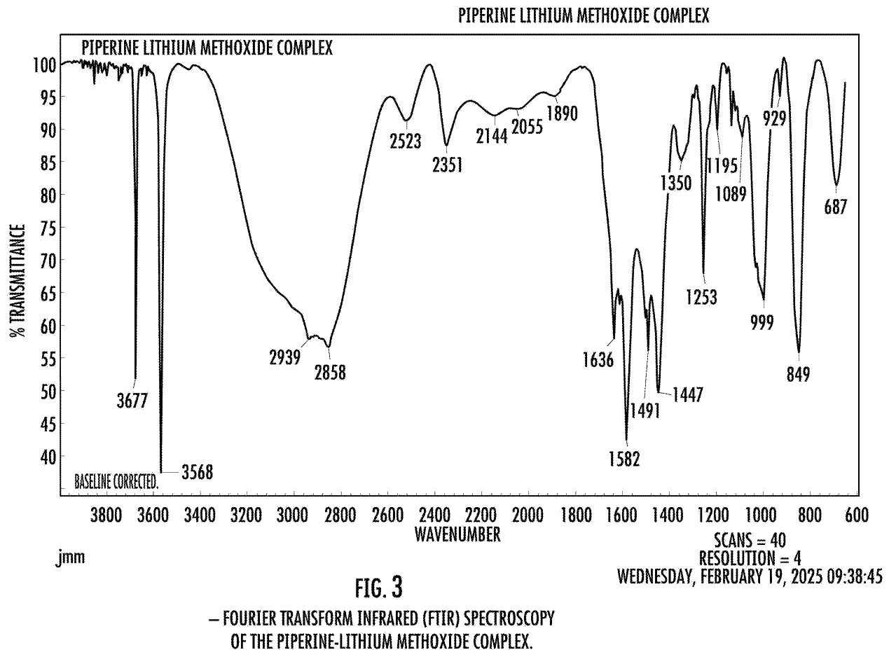

The orange solution was dried out under vacuum at 50° C. until a deep yellow powder was produced (FIG. 2).

1 gram of the product was completely dissolved in 500 ml of water after a few minutes of stirring. The Fourier Transform Infrared (FTIR) spectroscopy of the piperine-lithium methoxide complex is shown in FIG. 3.

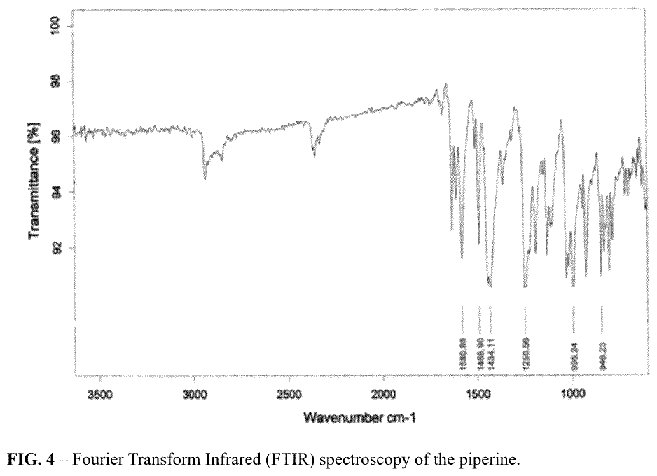

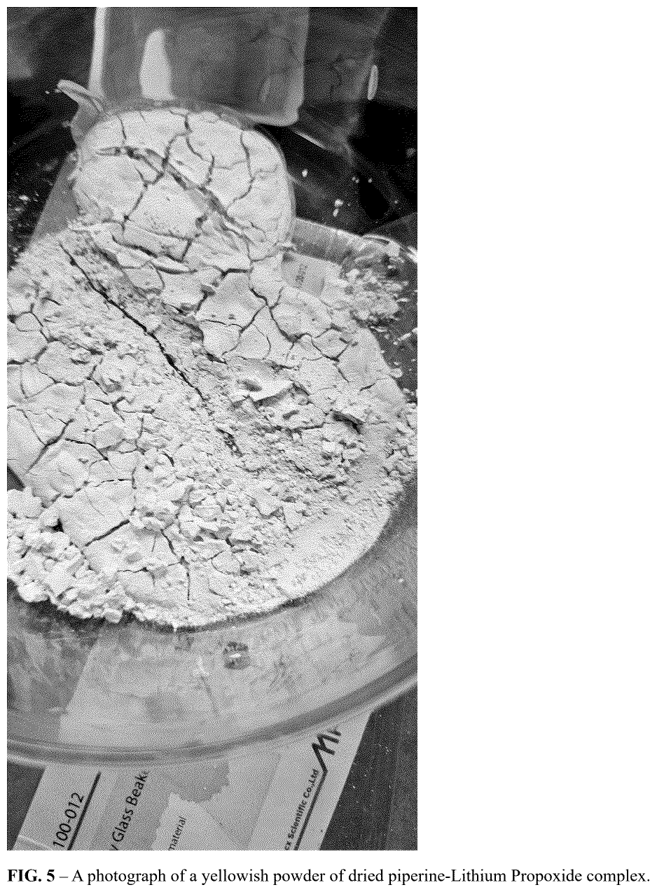

For comparison, FTIR spectroscopy of piperine is shown in FIG. 4. Piperine exhibits characteristic absorptions at approximately 1580 cm⁻¹, 1469 cm⁻¹, 1434 cm⁻¹, 1251 cm⁻¹, 995 cm⁻¹ and 846 cm⁻¹ (FIG. 4). In contrast, the dried piperine-lithium methoxide complex exhibits corresponding bands that are shifted and/or changed in relative intensity, including absorptions at approximately 1582 cm⁻¹, 1491 cm⁻¹, 1447 cm⁻¹, 1253 cm⁻¹, 999 cm⁻¹, and 849 cm⁻¹ (FIG. 3). The complex spectrum further shows additional absorptions not prominent in piperine alone, including bands in the ~3600-3500 cm−¹ region (e.g., ~3677 and ~3568 cm⁻¹) and additional bands in the fingerprint region (e.g., ~1350, ~1195, ~1089, and ~929 cm⁻¹). Collectively, these band shifts and new/altered absorptions are consistent with formation of a piperine-alkali metal alkoxide complex rather than piperine alone.

### Example 2—Preparation of Piperine-Lithium Propoxide Complex

150 ml of n-propanol were dried of water by stirring with 5 grams of molecular sieves for 1 hour. In the liquid, 3 grams of metallic Lithium were added, and the solution was left to stir for 24 hours till the Lithium dissolved. To the solution, 3 grams of piperine were added and stirred for 10 minutes. After filtering, the liquid was dried under vacuum at 70° C. until all n-propanol had evaporated. The result was a yellowish powder as shown in FIG. 5.

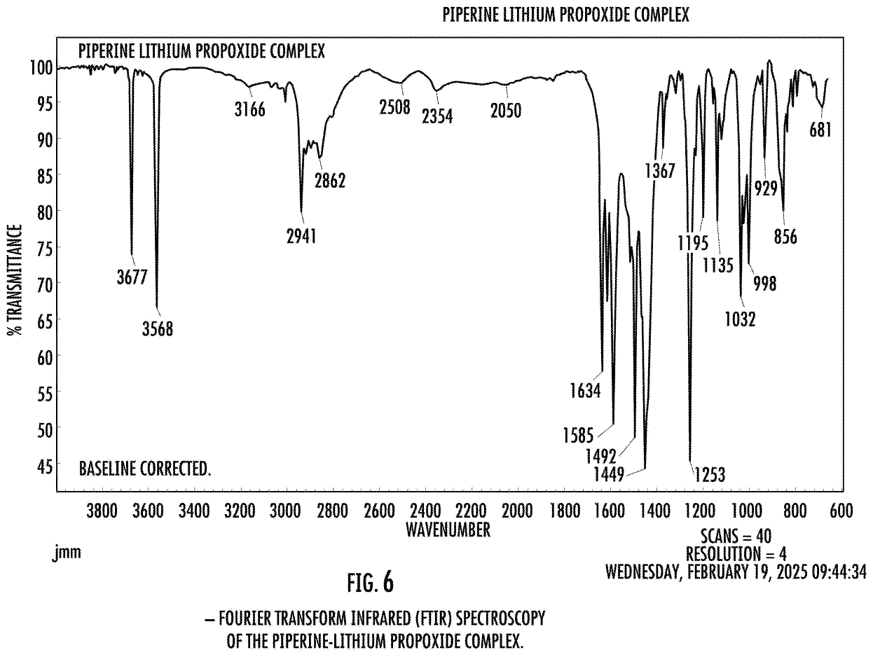

FTIR spectroscopy of the piperine-lithium propoxide complex is shown in FIG. 6. The complex exhibits characteristic absorptions including bands at approximately 1634 cm⁻¹, 1585 cm⁻¹, 1492 cm⁻¹, 1440 cm⁻¹, and 1253 cm⁻¹, together with additional bands in the fingerprint region (e.g., ~1195, ~1135, ~1032, ~968, ~929, and ~858 cm⁻¹) (FIG. 6). The spectrum further shows absorptions in the higher wavenumber region (e.g., ~3677 and ~3568 cm⁻¹, and a band near ~3168 cm⁻¹) (FIG. 6). By comparison to the FTIR spectrum of piperine (FIG. 4), the piperine-lithium propoxide complex shows band shifts and/or changes in relative intensity and additional absorptions, indicating that the isolated solid is spectroscopically distinct from piperine and is consistent with formation of a piperine-alkali metal alkoxide complex rather than piperine alone.

The powder exhibited good solubility with nearly all of the 1 gram of powder dissolving in 500 ml or room temperature water.

### Example 3—Preparation of Piperine Lithium Ethoxide Complex

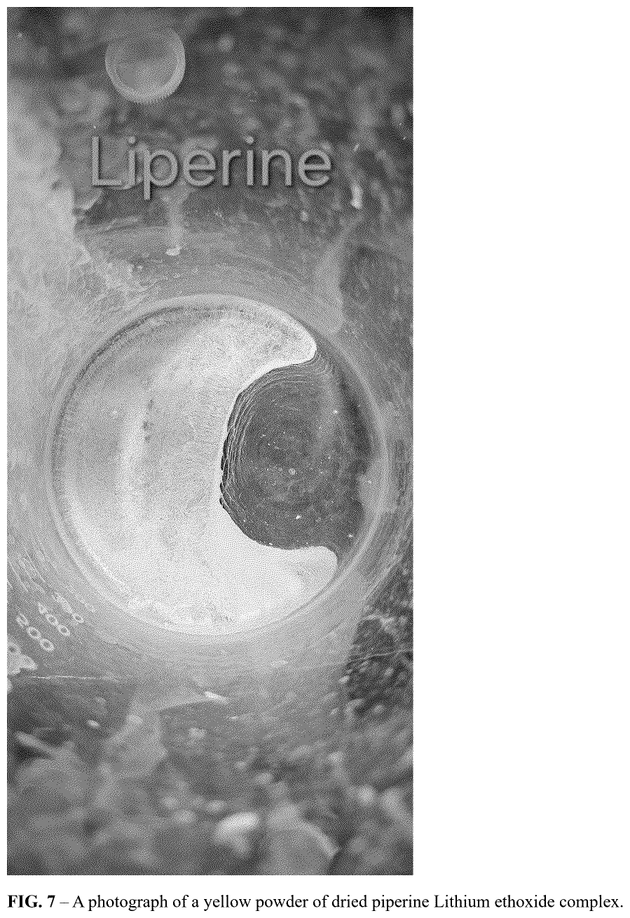

In 100 ml of absolute ethanol, 2 grams of Lithium metal were added. The solution was stirred briefly till all the metal dissolved. Then 2 grams of piperine were added and dissolved in the solution. The solution was dried under vacuum at 50 C. The product was a yellow powder as shown in FIG. 7. The calculated content of piperine by weight was 11.7%.

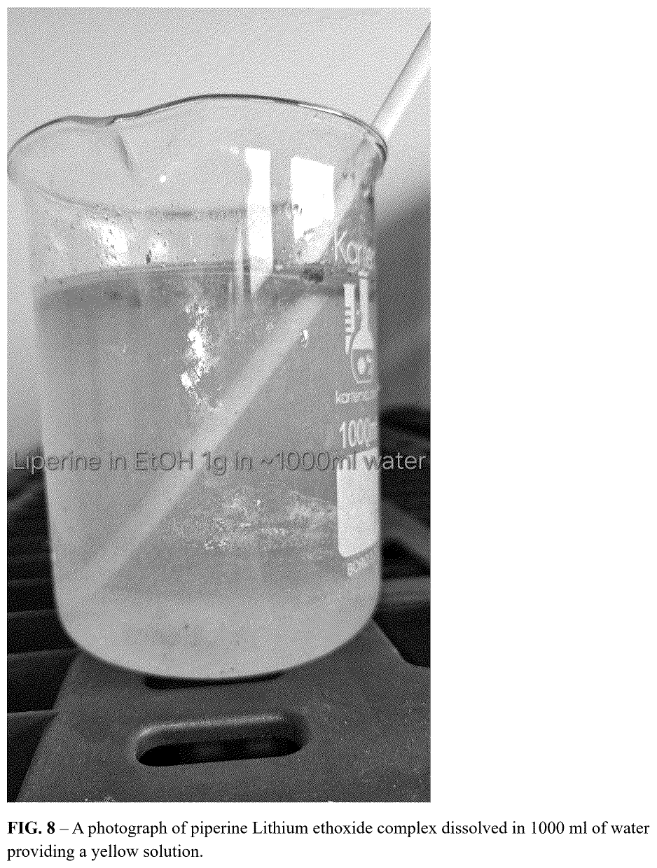

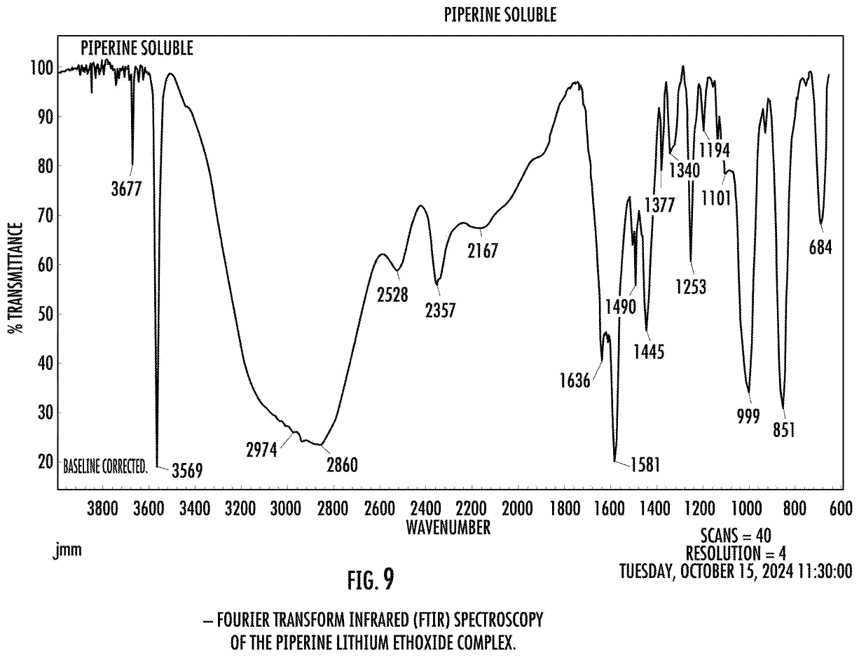

1 gram of the product nearly instantly dissolved in 1000 ml of water providing a yellow solution as shown in FIG. 8.

FTIR spectroscopy of the dried piperine-lithium ethoxide complex is shown in FIG. 9. The spectrum exhibits characteristic absorptions including bands at approximately 1581 cm⁻¹, 1445 cm⁻¹, and 1253 cm⁻¹, together with additional bands in the fingerprint region (e.g., ~1400, ~1377, ~1340, ~1194, ~1101, ~999, and ~851 cm⁻¹) (FIG. 9). The observed pattern of absorptions and relative intensities is consistent with a piperine-alkali metal alkoxide complex that is spectroscopically distinct from piperine alone.

### Example 4—Preparation of Piperine-Sodium Ethoxide Complex

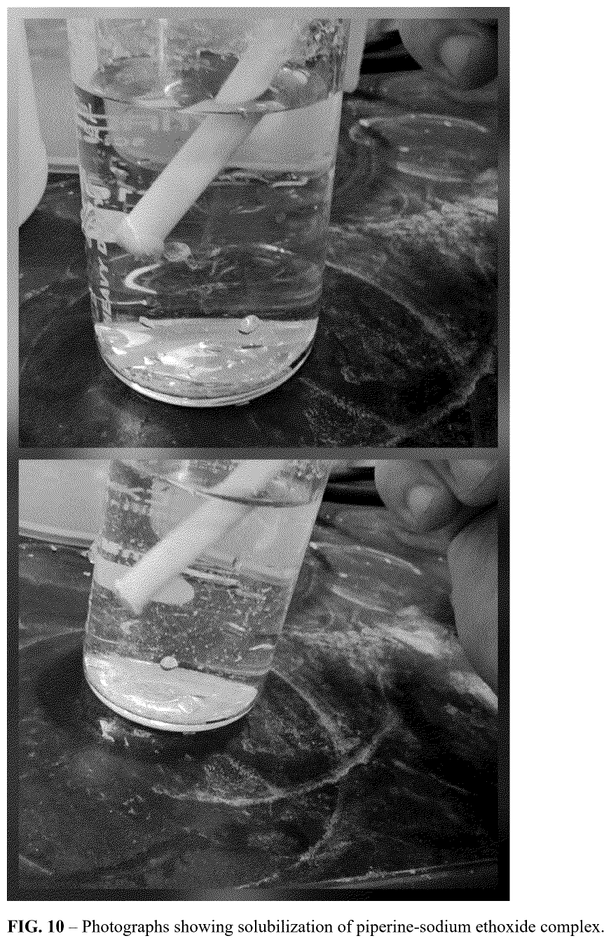

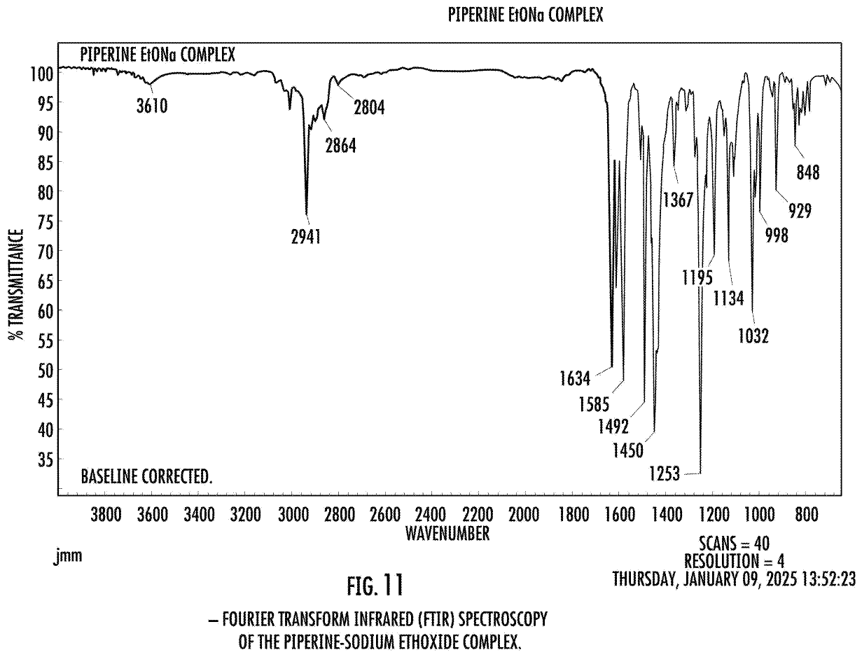

In 100 ml of absolute ethanol, 6 grams of sodium ethoxide powder were dissolved. Then 2 grams of piperine powder were added and dissolved. The solution was evaporated in vacuum at 50° C. The solution was dried at vacuum at 50° C. The product exhibited good solubility albeit lower than that of the piperine-lithium ethoxide complex (FIG. 10).

The FTIR of the compound was shown in FIG. 11.

### Example 5—Effect of Piperine and Piperine Lithium Ethoxide Complex in the Absorption of Amino Acids

To test the effectiveness of the new piperine-lithium ethoxide complex versus regular piperine, a 43 year old male performed the following experiments:

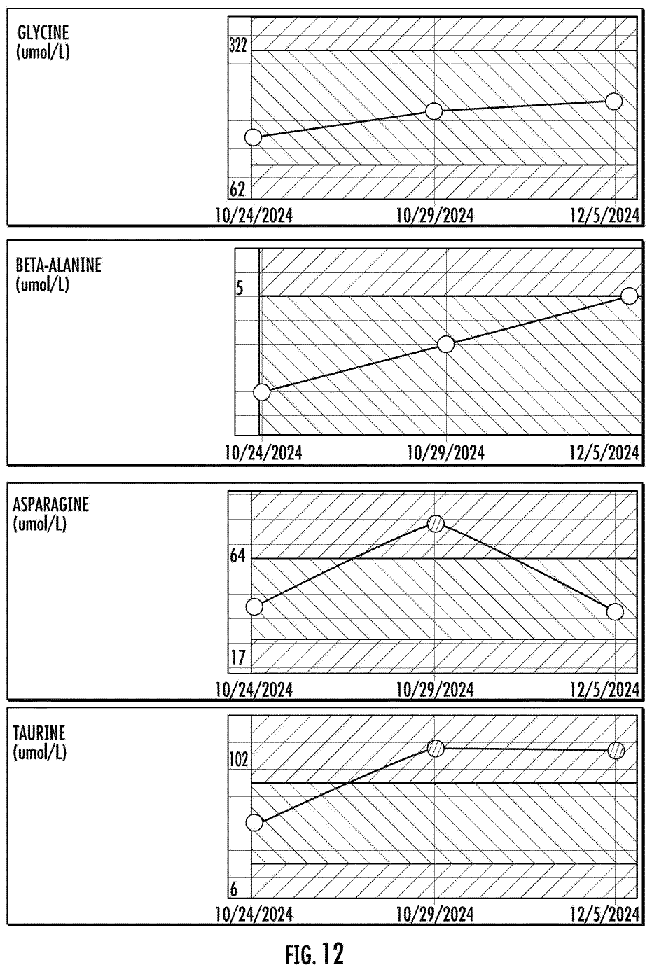

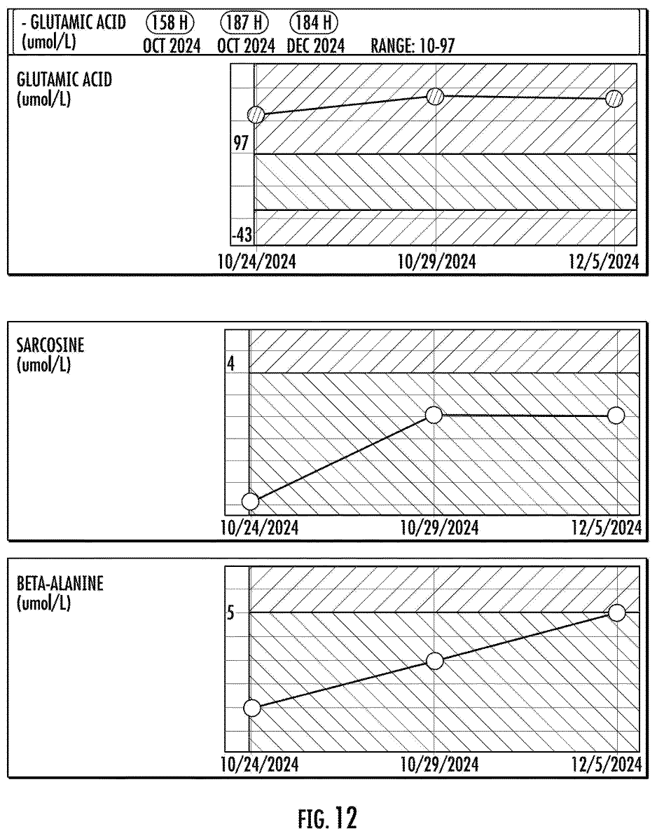

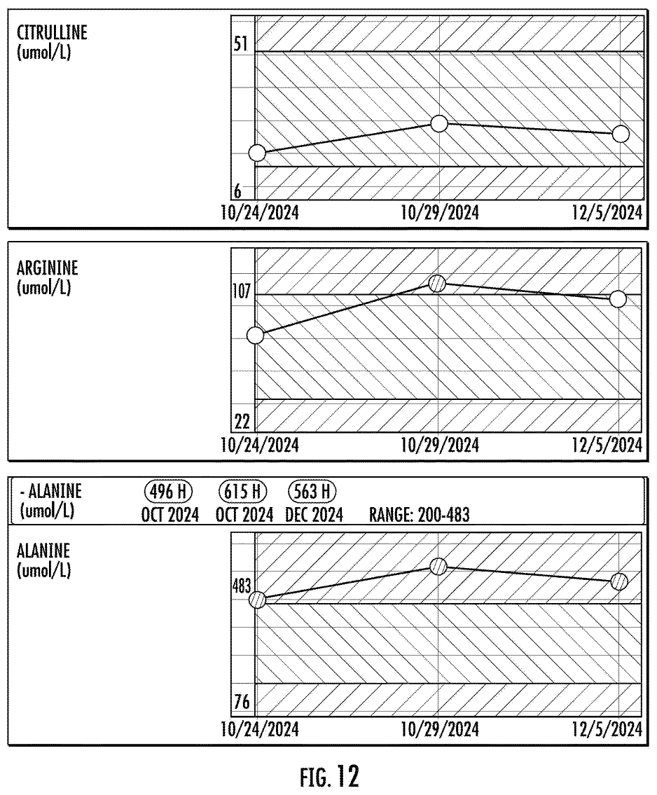

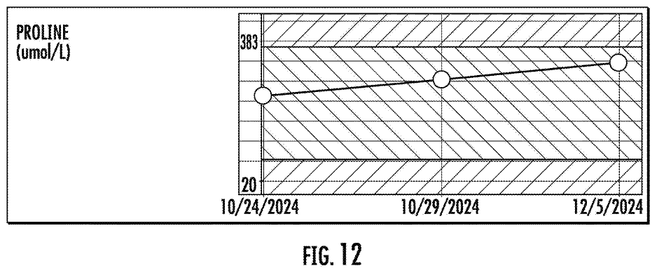

After fasting for 12 hours, he ingested 30 grams of whey protein in 500 ml water and then measured the levels of amino acids in the blood (10/24/24). After a few more days of washout he repeated the process but ingested 150 mg piperine-lithium ethoxide complex (10/29/24) (providing about 16 mg piperine, which is more than 5 times less than the dose of regular piperine used in the third step). After some days washout he repeated the experiment but instead ingested 100 mg of regular piperine together with the whey protein (12/4/24). The results on the absorption of the amino acids from the protein can be seen in FIG. 12.

As can be seen, despite the much lower dose of piperine, the piperine-lithium ethoxide complex produced an equal or even better increase in the absorption of amino acids than regular piperine. Those results come as highly surprising. To test the biological effects of the compounds, the same subject ingested either 500 mg of regular piperine or 1000 mg of the piperine-lithium ethoxide complex on fasted stomach. Regular piperine produced no notable effect except diarrhea for the rest of the day. The piperine-lithium ethoxide complex produced profound diaphoresis (sweating) which persisted for 6 hours. This further confirms the much higher bioavailability of the soluble complexes of piperine and also a reduction in unwanted side effects. In another word, administering piperine in the disclosed alkali metal alkoxide complex provides a greater enhancement of piperine bioavailability (and/or enhancement of bioavailability of a co-administered agent) than administering an equivalent amount of piperine in an uncomplexed form.

Various modifications and variations of the described methods, compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A composition comprising a complex of piperine and/or a metabolite or derivative thereof and an alkali metal alkoxide.

2. The composition of claim 1, wherein the alkali metal alkoxide is one of sodium ethoxide, potassium ethoxide, or lithium ethoxide.

3. The composition of claim 1, wherein the piperine and/or a metabolite or derivative thereof is piperine.

4. The composition of claim 1, wherein the weight ratio of alkali metal alkoxide to piperine and/or a metabolite or derivative thereof is between 1:0.1-1000:1.

5. The composition of claim 1, wherein the weight ratio of alkali metal alkoxide to piperine and/or a metabolite or derivative thereof is between 1:1 to 10:1.

6. A method of producing a composition comprising a complex of piperine and/or a metabolite or derivative thereof and an alkali metal alkoxide, the method comprising:
a) generating or dissolving an alkali metal alkoxide in an alcohol to form an alkali metal alkoxide alcohol solution;
b) dissolving piperine and/or a metabolite or derivative thereof in said alkali metal alkoxide alcohol solution to form a piperine and/or a metabolite or derivative thereof alkali metal alkoxide alcohol solution; and
c) drying said piperine and/or a metabolite or derivative thereof alkali metal alkoxide alcohol solution under anhydrous conditions.

7. The method of claim 6, wherein the alcohol is ethanol and the alkali metal alkoxide is an alkali metal ethoxide.

8. The method of claim 6, wherein the alkali metal alkoxide alcohol solution is obtained by dissolving an alkali metal ethoxide in an alcohol or by adding an alkali metal to an ethanol solution.

9. The method of claim 6, wherein the drying takes place at atmospheric humidity below 30%.

10. The method of claim 9, wherein the drying takes place at atmospheric humidity below 20%.

11. The method of claim 9, wherein the drying takes place at atmospheric humidity below 10%.

12. The method of claim 9, wherein the drying takes place in a vacuum.

13. The method of claim 9, wherein the anhydrous conditions consist of a water content below 1% in either gaseous or liquid compositions.

14. A method of improving athletic performance comprising administering to a subject 1-1000 mg of a composition comprising a complex of piperine and/or a metabolite or derivative thereof and an alkali metal alkoxide.

15. The method of claim 14, wherein the alkali metal alkoxide is one of sodium ethoxide, potassium ethoxide, or lithium ethoxide.

16. A method of improving the bioavailability and/or effectiveness of a dietary supplement, nutraceutical, or pharmaceutical comprising co-administering to a subject said supplement, nutraceutical, or pharmaceutical with a composition comprising a complex of piperine and/or a metabolite or derivative thereof and an alkali metal alkoxide.

17. The method of claim 16, wherein co-administering the dietary supplement, nutraceutical, or pharmaceutical with the composition results in a greater enhancement of bioavailability and/or effectiveness than administering an equivalent amount of piperine without the composition.

18. The method of claim 17, wherein the subject is administered between 1-1000 mg of the composition.
